(19) United States
(12) Patent Application Publication
(10) Pub. No.: US 2026/0104346 A1
(43) Pub. Date: Apr. 16, 2026

(54) An Experimental System for Rock Corrosion by Supercritical Carbon Dioxide

(71) Applicants: Shaoxing University, Shaoxing City (CN); China University of Mining and Technology, Xuzhou (CN)

(72) Inventors: Shengqi YANG, Shaoxing City (CN); Yue LI, Shaoxing City (CN); Yanhua HUANG, Shaoxing City (CN); Pengfei YIN, Shaoxing City (CN); Wenling TIAN, Shaoxing City (CN); Zhennan ZHU, Shaoxing City (CN)

(73) Assignees: Shaoxing University, Shaoxing City (CN); China University of Mining and Technology, Xuzhou (CN)

(21) Appl. No.: 19/420,413

(22) Filed: Dec. 15, 2025

(30) Foreign Application Priority Data
Jul. 7, 2025 (CN) 202510926442.7

(51) Int. Cl. G01N 17/00; G01N 33/24

(52) U.S. Cl. CPC G01N 17/002; G01N 33/24

(57) ABSTRACT

The present invention relates to an experimental system and usage method for supercritical carbon dioxide rock corrosion. The experimental system comprises a corrosion vessel for exposing samples to a saturated carbon dioxide aqueous solution. The inside of the corrosion vessel is provided with a heating assembly and a rock sample placement rack. The top of the rock sample placement rack is above the liquid level of saturated carbon dioxide aqueous solution, and the bottom of the rock sample placement rack is submerged in saturated carbon dioxide aqueous solution. The heating assembly is located on the perimeter wall of the corrosion vessel and connected with a controller. The corrosion vessel is connected to a carbon dioxide gas delivery unit and a booster pump assembly connected via a conveying pipe. The present invention enables rocks to corrode in both supercritical and carbon dioxide solutions.

### TECHNICAL FIELD

The present invention relates to the field of rock corrosion experimental technology, and more specifically, to an experimental system and method for testing the effects of supercritical carbon dioxide on rock corrosion.

### BACKGROUND

Supercritical carbon dioxide rock corrosion assays are used to study the physical and chemical properties of petrochemicals, mainly by testing the changes in chemical composition in rocks to analyze the strength performance of specimens. This type of experiment has a wide range of applications in materials science, chemical engineering, and geological exploration.

In a carbon dioxide geological sequestration environment, a rock/supercritical carbon dioxide/carbon dioxide groundwater solution exists in phase equilibrium. Supercritical carbon dioxide can dissolve certain minerals in rock, including carbonate rock, which may lead to changes in the physical and chemical properties of the rock. The carbon-dioxide groundwater solution will react with certain minerals in rock (such as silicates) and may form new mineral phases, which may have different physical and chemical properties, thus affecting the properties of the rock, Thus, it is necessary to study petrology while supercritical carbon dioxide-carbon dioxide groundwater solutions are in coexistence to provide empirical, reliable research data for materials science, chemical engineering and geological exploration. However, there are no device in existence that can provide a suitable environment for exposing rock samples to corrosion in supercritical phase and liquid phase environments at the same temperature and pressure. Accordingly it is of great significance to provide an experimental system to study the corrosion of rock in geological storage environments comprising supercritical carbon dioxide.

### SUMMARY

In view of the shortcomings of the above prior art, the purpose of the present invention is to provide an experimental system and usage method for supercritical carbon dioxide rock corrosion, which can corrode rocks in supercritical carbon dioxide and carbon dioxide solution at the same temperature and pressure state of a carbon dioxide geological storage environment.

In order to solve the above technical problems, the present invention adopts the following technical scheme:

The present invention provides an experimental system for supercritical carbon dioxide rock corrosion, comprising a corrosion vessel, wherein the corrosion vessel is used to store a saturated carbon dioxide aqueous solution. A top opening of the corrosion vessel is connected with a sealing cover, and the inside of the corrosion vessel is provided with a heating assembly and a rock sample placement rack. The top of the rock sample placement rack is located above the liquid level of the saturated carbon dioxide aqueous solution, and the bottom of the rock sample placement rack is submerged in the saturated carbon dioxide aqueous solution. The heating assembly is arranged on the perimeter wall of the corrosion vessel and is connected with a controller. The controller regulates the temperature of the heating assembly, and is used to make the temperature of the heating assembly suitable for maintaining a supercritical state of carbon dioxide.

The corrosion vessel is connected with a carbon dioxide gas conveying unit through a conveying pipe, and the carbon dioxide gas conveying unit comprises:

A gas source storage tank, used to supply carbon dioxide gas, wherein the gas source storage tank has a gas outlet;

A gas booster pump assembly, used to provide conditions for maintaining a supercritical state of carbon dioxide, wherein the gas booster pump assembly and gas outlet are connected through pipelines, and the gas booster pump assembly is connected to the conveying pipe.

Preferably, the gas booster pump assembly comprises a gas booster pump and a buffer vessel, with the buffer vessel arranged between the gas booster pump and the corrosion vessel, and the air inlet of the buffer vessel connected to the gas booster pump through a pipeline. The air outlet of the buffer vessel is connected to the corrosion vessel through a conveying pipe.

Preferably, the corrosion vessel and the buffer vessel are made of high-temperature and pressure-resistant anti-corrosion alloy.

Preferably, the conveyor pipe is provided with a pressure gauge and a needle pressure control valve for regulating the amount of carbon dioxide pressure.

Preferably, the corrosion vessel comprises a first corrosion chamber and a second corrosion chamber. Saturated carbon dioxide aqueous solution is stored in the first corrosion chamber and the second corrosion chamber, and the rock sample placement rack is set in each corrosion chamber. Each rock sample placement rack is arranged in the first corrosion chamber and the second corrosion chamber respectively, and the sealing cover comprises the first sealing cover and the second sealing cover. The first sealing cover is connected with the top open thread of the first corrosion chamber, the second sealing cover is connected with the bottom open thread of the second corrosion chamber via a transmission pipe and a conveyor pipe. The transmission pipe and the conveyor pipe are connected with the gas booster pump assembly through a pipeline, wherein the transmission pipe runs through the first sealing cover and is connected to the first corrosion chamber, and the transfer pipe is connected with the second corrosion chamber.

Preferably, the first sealing cover has a first sealing body connected with the first corrosion chamber thread, and the second sealing cover has a second sealing body connected with the second corrosion chamber thread, and the first sealing body and the second sealing body are provided with a first sealing ring and a second sealing ring on the upper and lower sleeves.

Preferably, the distance between the first sealing ring and the second sealing ring is 5cm˜5.2cm.

Preferably, all pipelines are provided with threaded interfaces at the inlet and outlet ends, and all pipelines are equipped with Teflon clad wires on the outside.

The present invention also gives a method for the use of an experimental system for supercritical carbon dioxide rock corrosion, comprising the following steps:

The rock sample is placed on the rock sample placement rack, and the corrosion vessel is filled with saturated carbon dioxide aqueous solution, so that the lower part of the rock sample is immersed in the saturated carbon dioxide aqueous solution.

Then the gas source storage tank is opened, and the carbon dioxide in the gas source storage tank is sent to the gas booster pump for pressurization. The pressurized carbon dioxide is sent into the corrosion vessel through the conveyor pipe.

The heating temperature of the heating component is controlled by the controller to convert the pressurized carbon dioxide gas sent into the corrosion vessel into supercritical carbon dioxide, so that the rock samples on the rock sample placement rack are placed in the supercritical phase and liquid phase environment.

The beneficial effects of the present invention compared with the prior art:

The experimental system for supercritical carbon dioxide rock corrosion given by the present invention can perform rock corrosion simulation experiments for the carbon dioxide geological storage environment, and solve the disadvantages that the existing devices cannot simulate the carbon dioxide geological storage environment for rock corrosion simulation experiments under the same temperature and pressure state.

During experiments, the carbon dioxide gas is pressurized by the gas booster pump assembly and sent into the corrosion vessel through the conveying pipe. In order to make the carbon dioxide feed into the corrosion vessel at a pressure of 7.38 MPa to reach the supercritical state, the temperature of the heating component is controlled by the controller, and the pressurized carbon dioxide sent into the corrosion vessel can reach the temperature of 31.1° C. in the corrosive vessel. Under these conditions when carbon dioxide reaches the supercritical state, the corrosion vessel realizes the simulation of the carbon dioxide geological storage environment. This system overcomes the shortcoming that devices in the existing technology cannot corrode the rock in the liquid phase and the supercritical carbon dioxide gas phase at the same time.

The buffer vessel is provided in the present invention not only for sending the high-pressure carbon dioxide gas boosted by the gas booster pump into the buffer vessel for storage, but also for ensuring that the pressurized carbon dioxide gas can be sent into the corrosion vessel at a stable pressure.

### BRIEF DESCRIPTION OF THE DRAWINGS

FIG. 1 is a schematic diagram of the overall structure of a supercritical carbon dioxide rock corrosion curing test device proposed by the present invention in a high temperature and high pressure environment.

FIG. 2 shows the structure of the corrosion vessel.

FIG. 3 is a schematic diagram of the structure of the high-pressure sealing cover.

FIG. 4 shows the structure of the buffer vessel.

FIG. 5 shows the schematic diagram of the gas booster pump structure (front).

FIG. 6 shows the schematic diagram of the gas booster pump structure (back).

FIG. 7 shows the schematic diagram of the carbon dioxide gas source structure.

FIG. 8 shows the structure of the controller, temperature controller, and resistive heating plate.

### REFERENCE NUMERALS

1. Corrosion vessel; 101. a second sealing cover; 102. a first pressure gauge; 103. a first needle pressure regulator; 2. the first sealing cover; 201. The first seal; 202. a first sealing ring; 203. a second sealing ring; 204. a second-needle pressure regulator; 205. a second pressure gauge; 206. The first inlet pipe; 207. High-pressure gas screw interface; 3. Buffer vessel; 301. a pressure buffer tank; 302. the first high-voltage interface screw; 303. a high-pressure gas three-way valve; 304. a second high-voltage interface screw; 305. a second entry pipe; 4. a gas booster pump; 401. a supercharged cylinder block; 402. a drive cylinder; 403. a low air pressure air inlet; 404. a third entry pipe; 405. a high-pressure air outlet; 5. an air source storage tank; 501. a gas pressure reducing valve; 502. an air source switch ball valve; 503. a high-concentration carbon dioxide cylinder; 6. Controller, 601, temperature controller, 602, resistance heating plate.

### DETAILED DESCRIPTION OF EMBODIMENTS

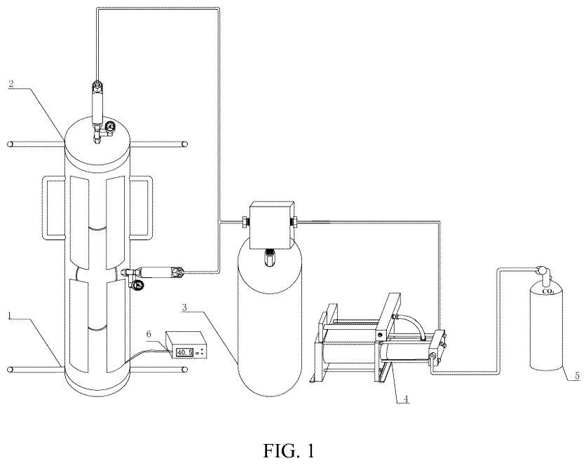

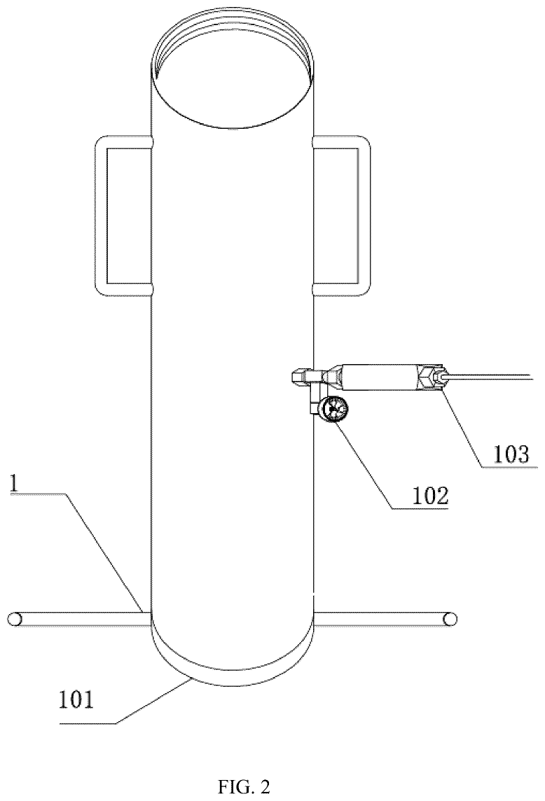

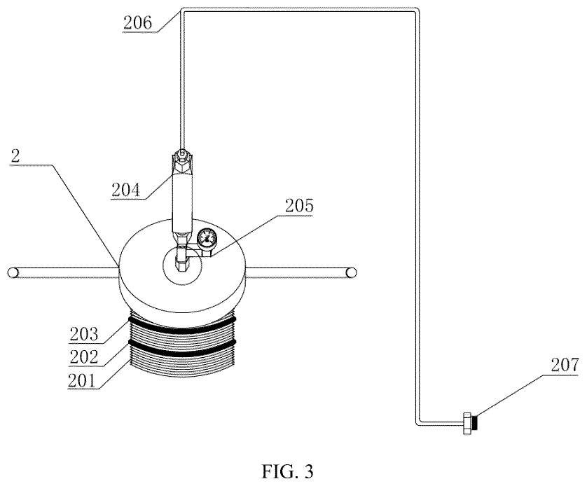

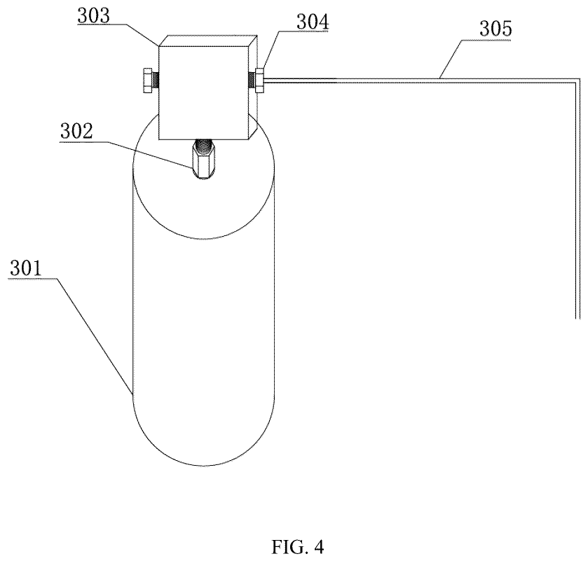

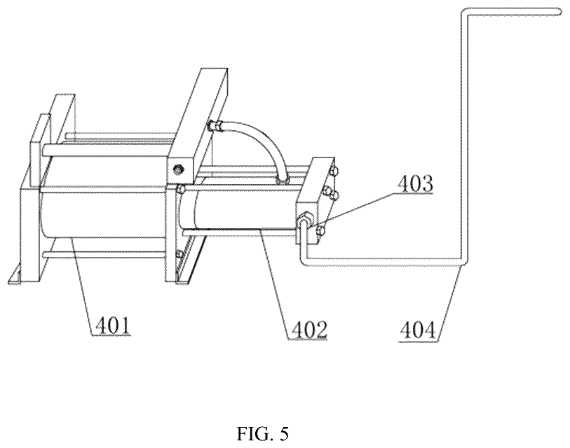

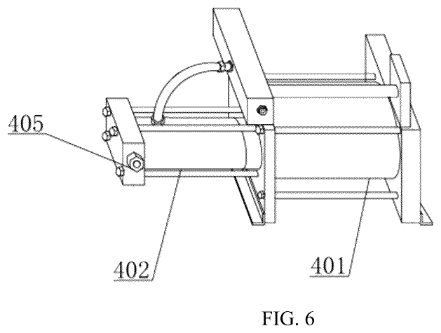

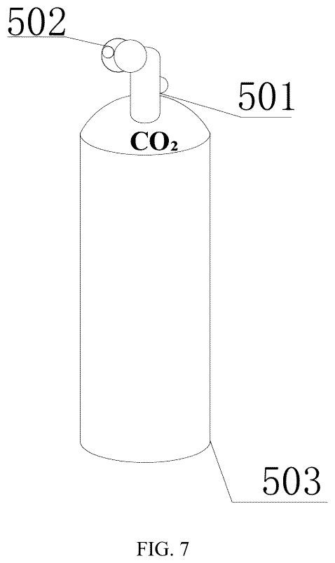

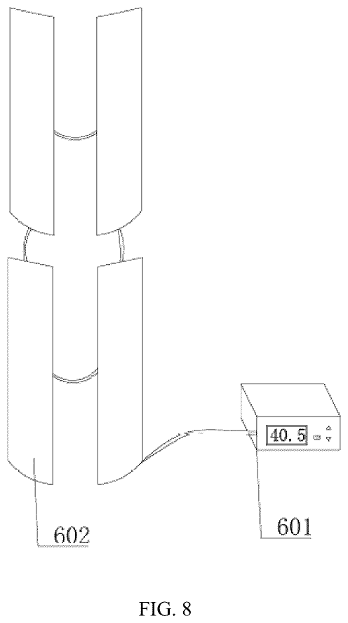

The following describes in detail the specific embodiments of the present invention, but it should be understood that the scope of protection of the present invention is not limited by the specific embodiments. Based on the embodiments of the present invention, all other embodiments obtained by persons of ordinary skill in the art without inventive effort are within the scope of protection of the present invention. The experimental methods described in the embodiments of the present invention are conventional methods unless otherwise specified. The inventors have discovered that existing rock corrosion experimental systems all conduct corrosion studies by immersing granite in a high-pressure reactor filled with supercritical carbon dioxide. The experimental device of the present invention primarily considers the coexistence of rock and supercritical CO₂—CO₂ groundwater solution in a CO₂ geological storage environment. Simultaneous corrosion experiments on rock in both environments are more realistic. To address the shortcomings of the prior art, the present invention aims to provide a supercritical CO₂ rock corrosion experimental system and method of use. This experimental system can simultaneously corrode rock in both a supercritical state and a CO₂ solution in a CO₂ geological storage environment. As shown in FIGS. 1 to 8, the present invention provides an experimental system for supercritical carbon dioxide rock corrosion, including a corrosion vessel 1, wherein the corrosion vessel 1 is used to store a saturated carbon dioxide aqueous solution, and the top of the corrosion vessel 1 is open and connected to a sealing cover, and a heating component and a rock sample placement rack are provided inside the corrosion vessel 1, wherein the top of the rock sample placement rack is located above the liquid surface of the saturated carbon dioxide aqueous solution, and the bottom of the rock sample placement rack is immersed in the saturated carbon dioxide aqueous solution, and the heating component is provided on the peripheral wall of the corrosion vessel 1 and connected to a controller 6, wherein the controller 6 regulates the temperature of the heating component so that the temperature of the heating component after regulation provides a temperature that maintains the supercritical state of carbon dioxide. Conditions are established to ensure that the carbon dioxide delivered into the corrosion vessel 1 reaches a supercritical temperature of 31.1° C. The corrosion vessel 1 is connected to a carbon dioxide gas delivery unit via a delivery pipe. The carbon dioxide gas delivery unit comprises:

A gas source tank 5 for supplying carbon dioxide gas and having a gas outlet. The gas source tank 5 comprises a high-concentration carbon dioxide cylinder 503, a gas pressure reducing valve 501, and a gas source on/off ball valve 502. A pipeline is connected to the top of the high-concentration carbon dioxide cylinder 503. The pipeline near the outlet is equipped with a gas source on/off ball valve 502 and a gas pressure reducing valve 501. The gas outlet of the gas source tank 5 is the outlet of the pipeline to which the gas source tank 5 is connected. A gas booster pump assembly is connected to the gas outlet (pipeline output) via a pipeline. The gas booster pump assembly is connected to the delivery pipe. The gas booster pump assembly ensures that the carbon dioxide delivered into the corrosion vessel 1 reaches a supercritical pressure of 7.38 MPa. The gas booster pump assembly includes a gas booster pump 4, which comprises a booster cylinder 401 and a drive cylinder 402. One end of the booster cylinder 401 is connected to the drive cylinder 402. One end of the drive cylinder 402 is provided with a low-pressure air inlet 403, which is connected to the gas source storage tank 5 via a third inlet pipe 404. The drive cylinder 402 is provided with a high-pressure air outlet 405, which is connected to the delivery pipe via a second inlet pipe 305. Specifically, the gas booster pump assembly includes the gas booster pump 4 and a buffer vessel 3. The buffer vessel 3 is located between the gas booster pump 4 and the corrosion vessel 1. The air inlet of the buffer vessel 3 is connected to the gas booster pump 4 via a pipeline, and the air outlet of the buffer vessel 3 is connected to the corrosion vessel 1 via a delivery pipe. Buffer vessel 3 comprises a pressure buffer tank 301, a first high-pressure interface screw 302, a high-pressure gas three-way valve 303, a second high-pressure interface screw 304, and a second inlet pipe 305. The top outlet of the pressure buffer tank 301 is connected to a pipeline via the first high-pressure interface screw 302, which is connected to one outlet of the high-pressure gas three-way valve 303. The second high-pressure interface screw 304 is located at one inlet of the high-pressure gas three-way valve 303 and is threadedly connected to the second inlet pipe 305. The other outlet of the high-pressure gas three-way valve 303 is connected to the corrosion vessel 1 via a delivery pipe. Specifically, both the corrosion vessel 1 and the buffer vessel 3 are made of a high-temperature, pressure-resistant, and corrosion-resistant alloy. This ensures that the materials of the buffer vessel 3 and the corrosion vessel 1 themselves are not corroded by CO₂ during long-term corrosion testing, which would otherwise contaminate the specimens with corrosion products. Both the buffer vessel 3 and the corrosion vessel 1 are made of a high-temperature, corrosion-resistant alloy, alloy numbered GH4161. Specifically, the delivery pipe is equipped with a pressure gauge and a needle-type pressure control valve for adjusting the CO₂ pressure. The pressure gauge includes a first pressure gauge 102 and a second pressure gauge 205, and the needle pressure control valve includes a first needle pressure regulating valve 103 and a second needle pressure regulating valve 204. This is intended to ensure that the buffer vessel 3 and corrosion vessel 1 materials themselves are not corroded by CO₂ during long-term corrosion testing, which would otherwise cause corrosion products from the metal materials to contaminate the specimen. Both the buffer vessel 3 and corrosion vessel 1 are made of a high-temperature corrosion-resistant alloy, alloy number GH4161. Specifically, the corrosion vessel 1 comprises two independent upper and lower first and second corrosion chambers, each containing a saturated carbon dioxide solution. At least two rock sample racks are provided, one in each of the first and second corrosion chambers. The sealing covers comprise a first sealing cover 2 and a second sealing cover 101. The first sealing cover 2 is threadedly connected to the top opening of the first corrosion chamber, while the second sealing cover 101 is threadedly connected to the bottom opening of the second corrosion chamber. The delivery pipe comprises a transmission pipe and a transmission pipe, both of which are connected to the gas booster pump assembly via pipelines. The transmission pipe is connected to the high-pressure gas screw interface 207 at the end of the first inlet pipe 206, passes through the first sealing cover 2, and connects to the first corrosion chamber. The transmission pipe is connected to the second corrosion chamber. The first inlet pipe 206 is also connected to a second needle pressure regulating valve 204 and a second pressure gauge 205, while the transmission pipe is also connected to the first needle pressure regulating valve 103 and a first pressure gauge 102. Specifically, the first sealing cover 2 has a first sealing body 201 threadedly connected to the first corrosion chamber, and the second sealing cover 101 has a second sealing body threadedly connected to the second corrosion chamber. A first sealing ring 202 and a second sealing ring 203 are sleeved above and below the first sealing body 201 and the second sealing body. Specifically, the distance between the first sealing ring 202 and the second sealing ring 203 is 5 cm to 5.2 cm. The first sealing ring 202 is positioned in the middle of the first sealing body 201 or the second sealing body, and the second sealing ring 203 is positioned at the threaded end of the first sealing body 201 or the second sealing body, to provide a tighter connection between the first sealing cover 2 and the first corrosion chamber, or between the second sealing cover 101 and the second corrosion chamber. Specifically, all pipelines have threaded connections at the inlet and outlet ends, and all pipelines are covered with polytetrafluoroethylene (PTFE) wire. The threads at the pipeline connections ensure connection and replacement if damaged. The PTFE wire protects the pipelines from CO₂ corrosion and is also wear-resistant. The present invention provides a method for using a supercritical carbon dioxide rock corrosion experimental system, comprising the following steps: Place a rock sample on a rock sample holder, and fill a corrosion vessel 1 with a saturated carbon dioxide aqueous solution so that the lower portion of the rock sample is immersed in the saturated carbon dioxide aqueous solution; Then, open a gas source tank 5, and pump the carbon dioxide in the gas source tank 5 into a gas booster pump 4 for pressurization. The pressurized carbon dioxide is then delivered to the corrosion vessel 1 through a delivery pipe. A controller 6 controls the heating temperature of a heating component to convert the pressurized carbon dioxide gas delivered to the corrosion vessel 1 into supercritical carbon dioxide, thereby placing the rock sample on the rock sample holder in a supercritical and liquid phase environment. Controller 6 includes a temperature controller 601 connected to a resistance heating element 602 for providing a constant temperature heat source for the corrosion vessel 1. The heating element is a resistance heating element 602. The desired temperature is set by the temperature controller 601, and the resistance heating element 602 provides a stable temperature. It is apparent that those skilled in the art may make various modifications and variations to the present invention without departing from the spirit and scope of the present invention. Thus, if these modifications and variations of the present invention fall within the scope of the claims of the present invention and their equivalents, the present invention is intended to include these modifications and variations.

What is claimed is:

1. An experimental system for assaying rock corrosion in supercritical carbon dioxide, comprising a corrosion vessel (1), wherein:
the corrosion vessel (1) contains saturated carbon dioxide aqueous solution;
a top opening of the corrosion vessel (1) is connected with a sealing cover;
the corrosion vessel (1) is provided with a heating assembly and contains a rock sample placement rack;
a top of the rock sample placement rack is located above the liquid level of the saturated carbon dioxide aqueous solution, and a bottom of the rock sample placement rack is submerged in the saturated carbon dioxide aqueous solution;
the heating assembly is arranged on a perimeter wall of the corrosion vessel (1) and connected with a controller (6);
the controller (6) regulates the temperature of the heating assembly, and is capable of regulating the temperature of the heating assembly to provide conditions for maintaining a carbon dioxide supercritical state;
the corrosion vessel (1) is connected with a carbon dioxide gas conveying unit through a conveying pipe, and the carbon dioxide gas conveying unit comprises:
a gas source storage tank (5) configured to supply carbon dioxide gas, wherein the gas source storage tank (5) has a gas outlet;
a gas booster pump assembly, configured to provide conditions for maintaining a supercritical state of carbon dioxide, wherein the gas booster pump assembly and the gas outlet are connected through a pipeline, and the gas booster pump assembly is connected to the conveying pipe;
the corrosion vessel (1) comprises two independent corrosion chambers, a first corrosion chamber and a second corrosion chamber, and the first corrosion chamber and the second corrosion chamber are both configured to contain saturated carbon dioxide aqueous solution;
at least two rock sample placement racks are provided, and each rock sample placement rack is respectively provided in the first corrosion chamber and the second corrosion chamber;
the sealing cover includes a first sealing cover (2) and a second sealing cover (101);
the first sealing cover (2) is threadedly connected to the top opening of the first corrosion chamber, and the second sealing cover (101) is threadedly connected to the bottom opening of the second corrosion chamber;
the delivery pipe includes a first transmission pipe and a second transmission pipe;
the first transmission pipe and the second transmission pipe are both connected to the gas booster pump assembly through a pipeline;
the first transmission pipe passes through the first sealing cover (2) and is connected to the first corrosion chamber, and the second transmission pipe is connected to the second corrosion chamber.

2. The experimental system for assaying rock corrosion in supercritical carbon dioxide according to claim 1, wherein the gas booster pump assembly comprises a gas booster pump (4) and a buffer vessel (3);
wherein the buffer vessel (3) is arranged between the gas booster pump (4) and the corrosion vessel (1), and the air inlet of the buffer vessel (3) is connected to the gas booster pump (4) through a pipeline, and the air outlet of the buffer vessel (3) is connected to the corrosion vessel (1) through a conveying pipe.

3. The experimental system for assaying rock corrosion in supercritical carbon dioxide according to claim 2, wherein the corrosion vessel (1) and the buffer vessel (3) are both made of high-temperature and pressure-resistant anti-corrosion alloy.

4. The experimental system for rock corrosion in supercritical carbon dioxide according to claim 1, wherein a pressure gauge and a needle pressure control valve configured to regulate carbon dioxide pressure levels are provided on the conveyor pipe.

5. The experimental system for assaying rock corrosion in supercritical carbon dioxide according to claim 1, wherein the first sealing cover (2) has a first sealing body (201) threaded with the first corrosion chamber, the second sealing cover (101) has a second sealing body connected with the second corrosion chamber thread, and the first sealing body (201) and the second sealing body are provided with a first sealing ring (202) and a second sealing ring (203) on the upper and lower sleeves.

6. The experimental system for assaying rock corrosion in supercritical carbon dioxide according to claim 1, wherein the distance between the first sealing ring (202) and the second sealing ring (203) is 5cm˜5.2cm.

7. The experimental system for assaying rock corrosion in supercritical carbon dioxide according to claim 1, wherein all pipelines are provided with threaded interfaces at the inlet and outlet ends, and all pipelines are equipped with teflon clad wires on the outside.

8. A method of using the experimental system for assaying rock corrosion in supercritical carbon dioxide according to claim 1, comprising the following steps:
a rock sample is placed on the rock sample placement rack, and the saturated carbon dioxide aqueous solution is filled into the corrosion vessel (1), so that the lower part of the rock sample is immersed in the saturated carbon dioxide aqueous solution;
the gas source storage tank (5) is opened, sending the carbon dioxide in the gas source storage tank (5) into the gas booster pump (4) for pressurization, and the pressurized carbon dioxide is sent into the corrosion vessel (1) through the conveyor pipe;
the heating temperature of the heating assembly is adjusted by the controller to convert the pressurized carbon dioxide gas fed into the corrosion vessel (1) into supercritical carbon dioxide, so that the rock samples on the rock sample placement rack are placed in the supercritical phase and liquid phase environment.
